(19) United States
(12) Patent Application Publication
(10) Pub. No.: US 2016/0331941 A1
(43) Pub. Date: Nov. 17, 2016

(54) DILATOR

(71) Applicant: YUGEN KAISHA OKID, Syuchi-gun (JP)

(72) Inventor: Yosuke OKADA, Syuchi-gun (JP)

(21) Appl. No.: 14/883,947

(22) Filed: Oct. 15, 2015

(30) Foreign Application Priority Data
May 11, 2015 (JP) 2015-096133

(51) Int. Cl. A61M 25/06; A61M 25/09

(52) U.S. Cl. CPC A61M 25/0662; A61M 25/09

(57) ABSTRACT

The invention provides a dilator having characteristics as an inexpensive instrument that allows insertion of a guidewire for assisting insertion of a catheter into a vessel in a simple and smooth manner. A first through-hole 5 into which a guidewire can be inserted is pierced over an entire region of a dilator 1 from a distal end face 3a to a rear end face 3b in the longitudinal direction of the dilator. A second through-hole 7 into which a guidewire can be inserted is pierced over a region of the dilator 1 from a midway to the rear end face 3b in the longitudinal direction of the dilator. A tapered portion 9 is formed such that it is tapered toward the distal end face 3a. An opening 7a a of the second through-hole 7 is open on the surface of the tapered portion 9.

### BACKGROUND OF THE INVENTION

1. Field of the Invention

The present invention relates to a dilator for assisting subcutaneous insertion of a catheter with two fluid passages into a vessel via an insertion puncture.

2. Description of the Related Art

U.S. 2014/0088510 A describes subcutaneous insertion of a catheter system 10 with two internal lumens, or fluid passages, into a vessel 4, wherein two guidewires 26A and 26B are inserted into the vessel 4 prior to the insertion of the catheter system 10 such that two distal tip portions 18A and 18B of the catheter system 10 are respectively inserted into the vessel 4 via the guidewires 26A and 26B.

However, a method for inserting the first guidewire 26A and the second guidewire 26B into the vessel 4 is not detailed in U.S. 2014/0088510 A. As described in [Related Art] of JP 2002-291902 A, the insertion method includes, for example, a method using an outer tube having a puncture needle inserted therein and an introducer, which is a set of a sheath and a dilator inserted therein. It is also well known in the art to use a relatively thick hollow needle as the puncture needle to omit the use of the outer tube. In such a case, the procedures for inserting the guidewires and the catheter are roughly as follows. First, a tip of a puncture needle is inserted into a vessel 4 via an insertion puncture in the skin formed by incision, and in this state, the first guidewire 26A is inserted into the vessel 4 via the puncture needle. Then, the puncture needle is pulled out from the first guidewire 26A. Next, the introducer is inserted into the vessel 4 via the first guidewire 26A. After pulling out the dilator from the introducer inserted into the vessel 4, the second guidewire 26B is inserted into the sheath remaining in the vessel 4 so as to insert the second guidewire 26B into the vessel 4. Then, the sheath is pulled out from both the guidewires 26A and 26B, and the two distal tip portions 18A and 18B of the catheter 10 are respectively inserted into the vessel 4 via both the guidewires 26A and 26B remaining in the vessel 4.

### SUMMARY OF THE INVENTION

However, with the conventional method of inserting two guidewires into the vessel through one insertion puncture formed in the skin, a step between an outer peripheral portion of the dilator projecting from the sheath tip and an outer peripheral portion of the sheath tip made resistance when inserting the introducer while dilating the insertion puncture, and thus the introducer could not be inserted smoothly.

Further, with the conventional method, the dilator had to be pulled out from the introducer when inserting the second guidewire, and this was a complicated task.

Also, because only one through-hole was formed in the sheath, there was a chance that the secondly inserted second guidewire 26B becomes kinked with the firstly inserted first guidewire 26A which is already in the through-hole when the second guidewire 26B is inserted into the through-hole.

Still further, the introducer used for inserting the secondly inserted guidewire in the conventional method was very expensive, since the introducer was configured as a set of a sheath and a dilator inserted therein.

The present invention has been made in view of such drawbacks, and therefore the purpose of the invention is to provide a dilator having characteristics as an inexpensive instrument that allows insertion of a guidewire for assisting insertion of a catheter into a vessel in a simple and smooth manner.

To achieve this purpose, the dilator according to the present invention is a stem-like dilator for assisting insertion of a catheter into a vessel, including: a first through-hole pierced over an entire region of the dilator from a distal end face to a rear end face in a longitudinal direction of the dilator; a second through-hole, which is a different through-hole from the first through-hole, pierced over a region of the dilator from a midway to the rear end face in the longitudinal direction of the dilator; and a tapered portion formed such that it is tapered toward the distal end face, in which the first through-hole and the second through-hole are sized to allow insertion of flexible guidewires for assisting insertion of the catheter.

A dilator according to claim 2 of the invention is the dilator according to claim 1, in which one end of the second through-hole located at the midway of the dilator in the longitudinal direction thereof is open on a surface of the tapered portion while the other end of the second through-hole is open on the rear end face.

A dilator according to claim 3 of the invention is the dilator according to claim 1 or 2, in which when seen from a direction along the axis of the dilator, an opening in the one end of the second through-hole and an opening in the other end of the second through-hole are biased to the same direction with respect to the axis of the dilator.

A dilator according to claim 4 of the invention is the dilator according to any one of claims 1 to 3, in which the opening in the rear end face of the first through-hole is different from the opening in the rear end face of the second through-hole in at least one of the shape and the size thereof.

A dilator according to claim 5 of the invention is the dilator according to any one of claims 1 to 4, in which a mark that is observable from outside is indicated on a region including at least one of the rear end face and the proximity of the rear end face, and a positional relationship between a position of the opening on one end of the second through-hole located at the midway of the dilator in the longitudinal direction thereof and a position indicated by the mark is correlated in advance with the direction about the axis of the dilator.

A dilator according to claim 6 of the invention is the dilator according to any one of claims 1 to 5, in which a recessed portion is formed by recessing a part of the surface of the dilator excluding the distal end face and the rear end face, and the one end of the second through-hole is open in the recessed portion.

A dilator according to claim 7 of the invention is the dilator according to claim 6, wherein the recessed portion is formed as a long groove extending in a direction along the axis of the dilator, and a plurality of recessed portions are formed in the surface of the dilator at a certain interval in a circumferential direction thereof, and the one end of the second through-hole is open in one of the plurality of recessed portions.

According to the invention described in claim 1, because the first through-hole and the second through-hole are separately pierced in the dilator, the two guidewires can be inserted into the through-holes, respectively. Accordingly, when the secondly inserted guidewire is inserted into the through-hole of the dilator, the secondly inserted guidewire can be smoothly inserted until it is inside the vessel without being kinked with the firstly inserted guidewire that is inserted inside the through-hole in advance or involved in other incidents that hinder insertion of the secondly inserted guidewire.

Also, the dilator, which has been inserted until it is inside the vessel for dilating the opening in the skin or the opening in the vessel to facilitate insertion of the catheter, can be used as is in this state to insert the secondly inserted guidewire until it is inside the vessel. Thus, it is possible to insert the secondly inserted guidewire by using the dilator without having to use the conventional introducer which is a set of a sheath and a dilator inserted therein. As a result, instrumental cost for insertion of the guidewire will be reduced, and at the same time, the insertion operation of the guidewire will be simplified due to the elimination of the operation for pulling out the dilator from the introducer. Further, because a conventional type introducer is not used, resistance due the step between the sheath and the dilator is not induced, and thus the dilator can be smoothly inserted into the opening in the skin and the opening in the vessel.

Further, because the dilator has a tapered portion tapered toward the distal end face, the dilator can be easily inserted into the opening in the skin and into the opening in the vessel in which the firstly inserted guidewire is inserted, and at the same time the dilator can dilate the opening in the skin or the opening in the vessel by the tapered portion as the dilator is advanced, which allows the dilator to be smoothly inserted into the opening in the skin and the opening in the vessel.

According to the invention described in claim 2, because one end of the second through-hole located at the midway of the dilator in the longitudinal direction thereof is open on the surface of the tapered portion, the diameter of the dilator at which the opening is located is thinner than parts other than the tapered portion. Accordingly, a gap tends to form between the part of the opening of the second through-hole and the inner wall of the vessel, and the secondly inserted guidewire is less likely to damage the vessel when the secondly inserted guidewire is inserted into the vessel via the second through-hole. Also, even if a gap is not formed between the part of the opening of the second through-hole and the inner wall of the vessel, adhesion force between the part of the opening of the second through-hole and the inner wall of the vessel can be reduced because the dilator is thinned at the part of the opening. As a result, it is less likely to damage the vessel by the secondly inserted guidewire when the secondly inserted guidewire is inserted into the vessel via the second through-hole.

According to the invention described in claim 3, because the opening on one end and the opening on the other end of the second through-hole are provided biased to the same direction with respect to the axis of the dilator, when the dilator is inserted until it is inside the vessel, the insertion is conducted by visually checking such that the position of the opening of the second through-hole in the rear end face of the dilator is at a desired position, and accordingly, the opening of the second through-hole located inside the vessel can be positioned at a desired position inside the vessel in a circumferential direction thereof.

According to the invention described in claim 4, because at least one of the shape and the size of the respective openings of the first through-hole and the second through-hole in the rear end face of the dilator are different from each other, the first through-hole can be easily distinguished from the second through-hole by merely looking at the respective openings.

According to the invention described in claim 5, because the relationship between the position of the opening on one end of the second through-hole located at the midway of the dilator in the longitudinal direction thereof and the position indicated by the mark is correlated in advance with the direction about the axis of the dilator, by visually checking such that the position of the mark is at a desired position when inserting the dilator until it is inside the vessel, the opening of the second through-hole located inside the vessel can be positioned at desired position inside the vessel in the circumferential direction thereof.

According to the invention described in claim 6, because the recessed portion is formed by recessing a part of the surface of the dilator excluding the distal end face and the rear end face, and the one end of the second through-hole is open in the recessed portion, it is less likely to damage the inner wall of the vessel by corner portions of the opening.

According to the invention described in claim 7, because the recessed portion is formed as a long groove extending in a direction along the axis of the dilator and because a plurality of recessed portions are formed in the surface of the dilator at a certain distance apart from each other in the circumferential direction thereof, a contact area between the tapered portion of the dilator and the opening in the skin and the opening in the vessel becomes small at the time when the tapered portion of the dilator is inserted into the opening in the skin or the opening in the vessel. Consequently, the resistance induced during insertion of the tapered portion of the dilator into the opening in the skin and the opening in the vessel can be reduced.

### BRIEF DESCRIPTION OF THE DRAWINGS

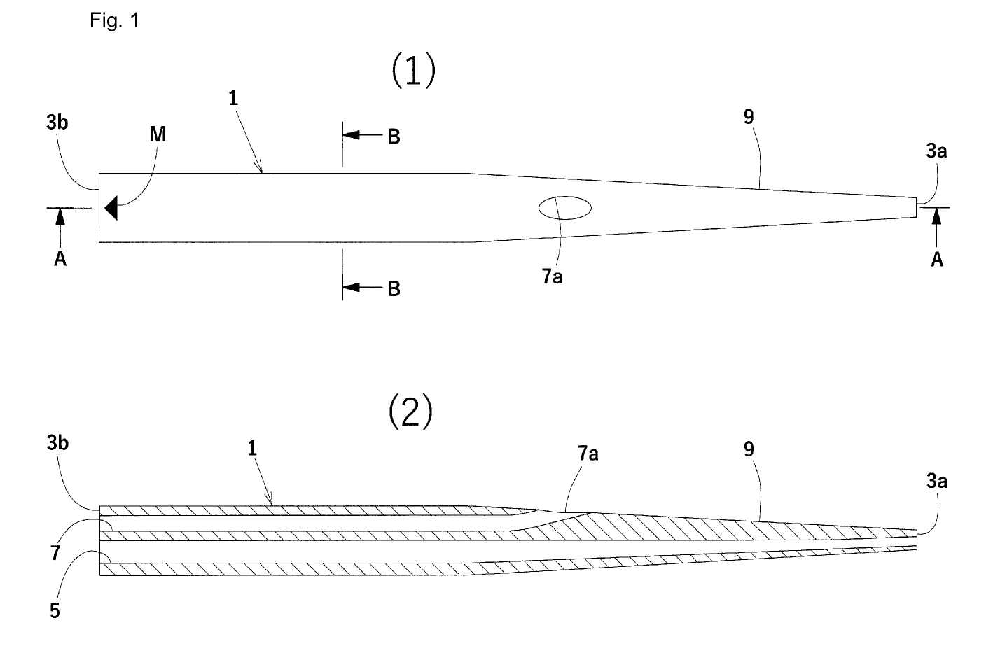

In FIG. 1, (1) is a side view, showing a dilator according to a first embodiment of the present invention and (2) is a sectional view along a line A-A in (1).

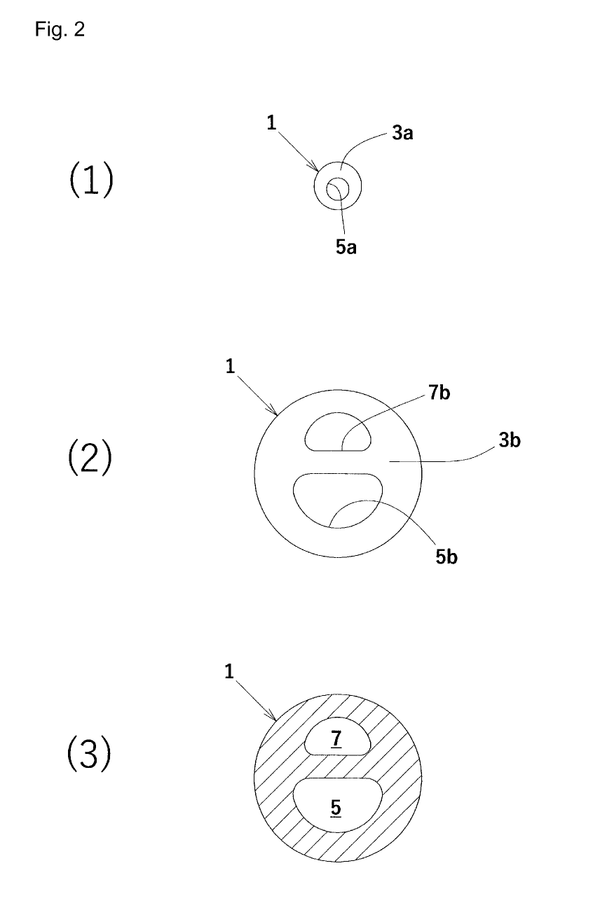

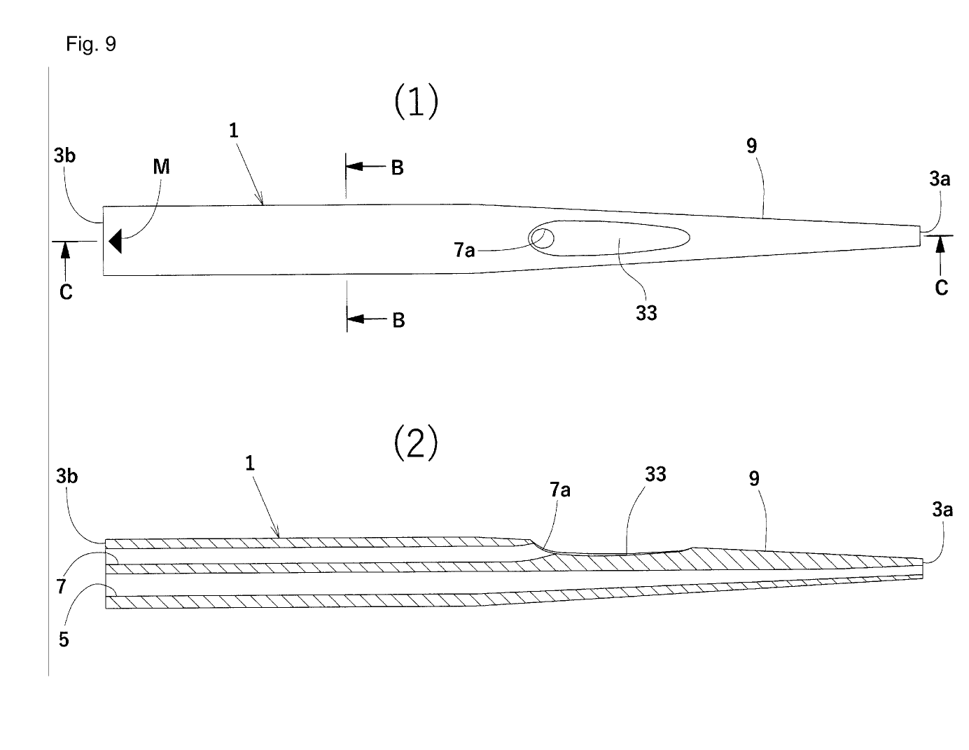

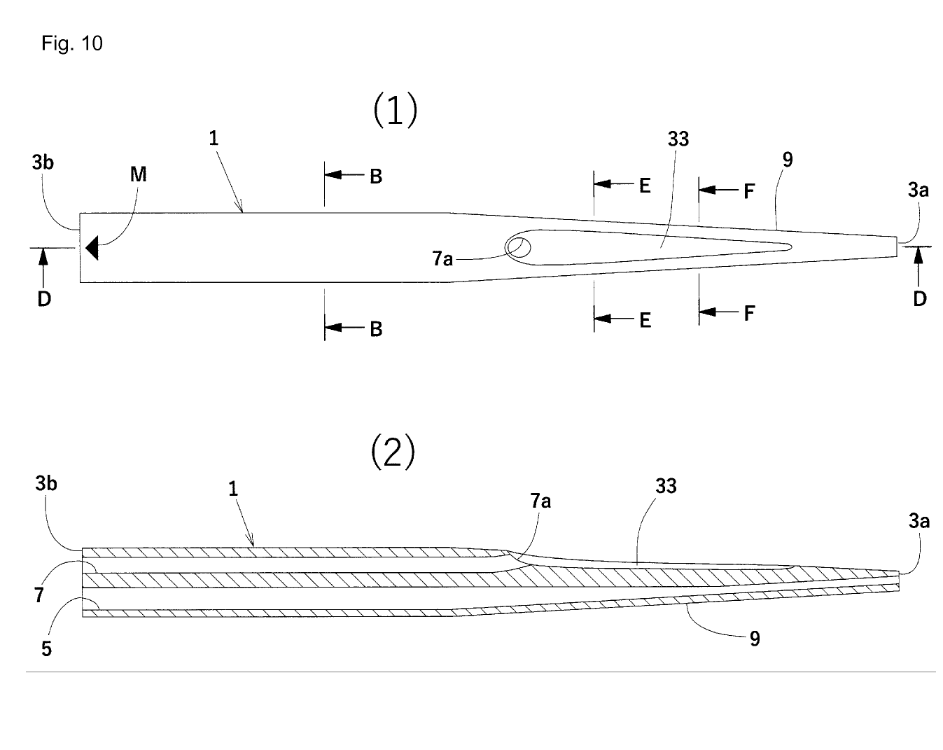

In FIG. 2, (1) and (2) are enlarged end views of end faces of dilators according to the first embodiment to a third embodiment of the present invention, the former end view showing a distal end face and the latter showing a rear end face, as seen from an opposite direction thereof, respectively, and (3) is an enlarged sectional view along line B-B in (1) of FIG. 1, as well as in (1) of FIG. 9 and (1) of FIG. 10, which will be described later.

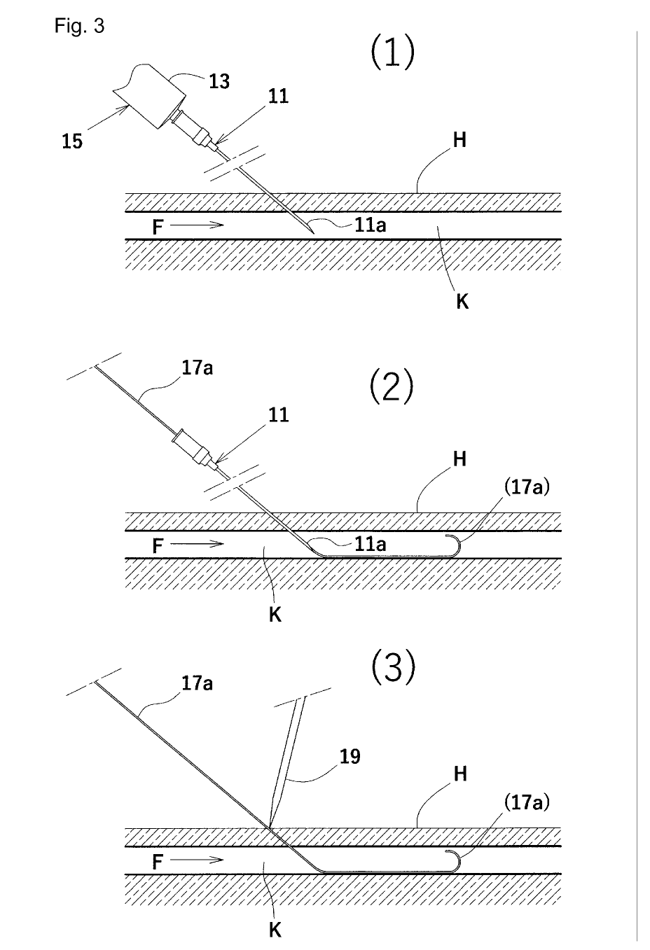

In FIG. 3, (1) is a view showing a state where a puncture needle of a syringe is penetrated through the skin to be inserted into a vessel, (2) is a view showing a state where a guidewire is inserted into a hollow space inside the puncture needle and advanced until the guidewire is inside the vessel, and (3) is a view showing a state where a puncture in the skin formed by the puncture needle is dilated by a skin-cutting scalpel.

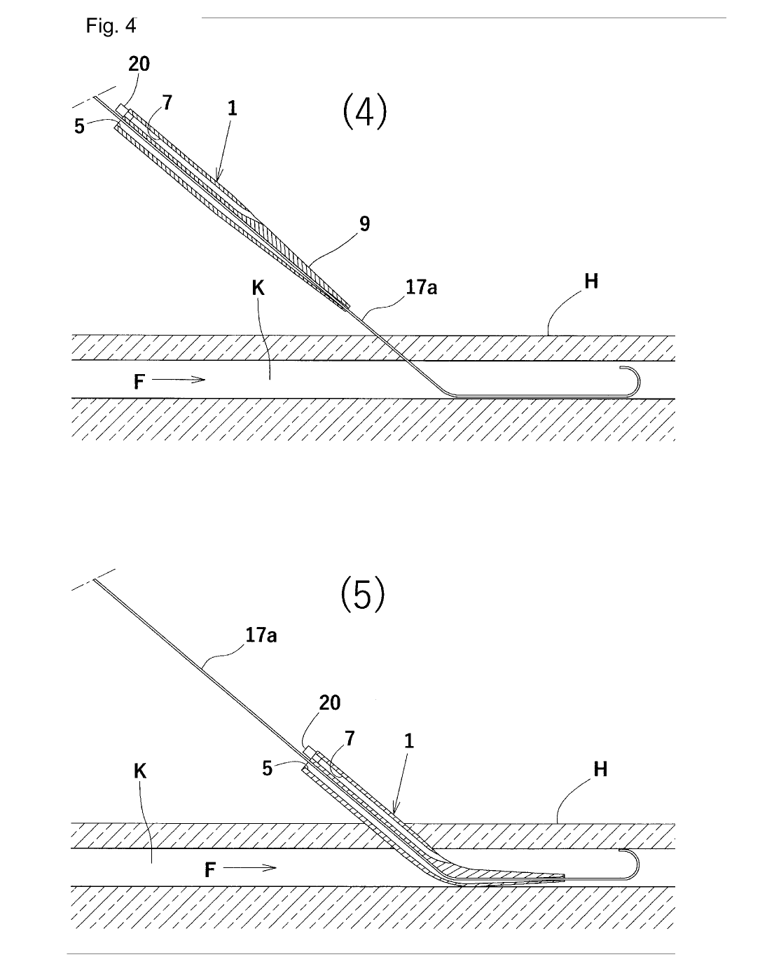

In FIG. 4, (4) is a view showing a state where the dilator is stuck over the guidewire, and (5) is a view showing a state where the dilator is inserted until it is inside the vessel.

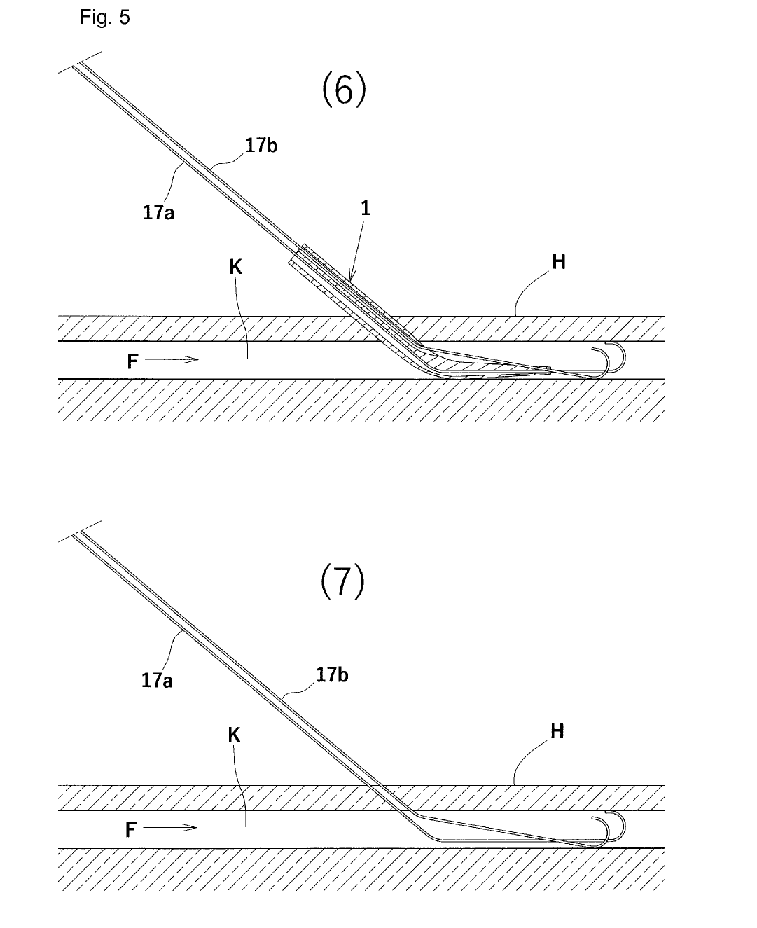

In FIG. 5, (6) is a view showing a state where a secondly inserted guidewire is inserted into the dilator and the guidewire is advanced until it is inside the vessel, and (7) is a view showing a state where the dilator is pulled out from both the guidewires.

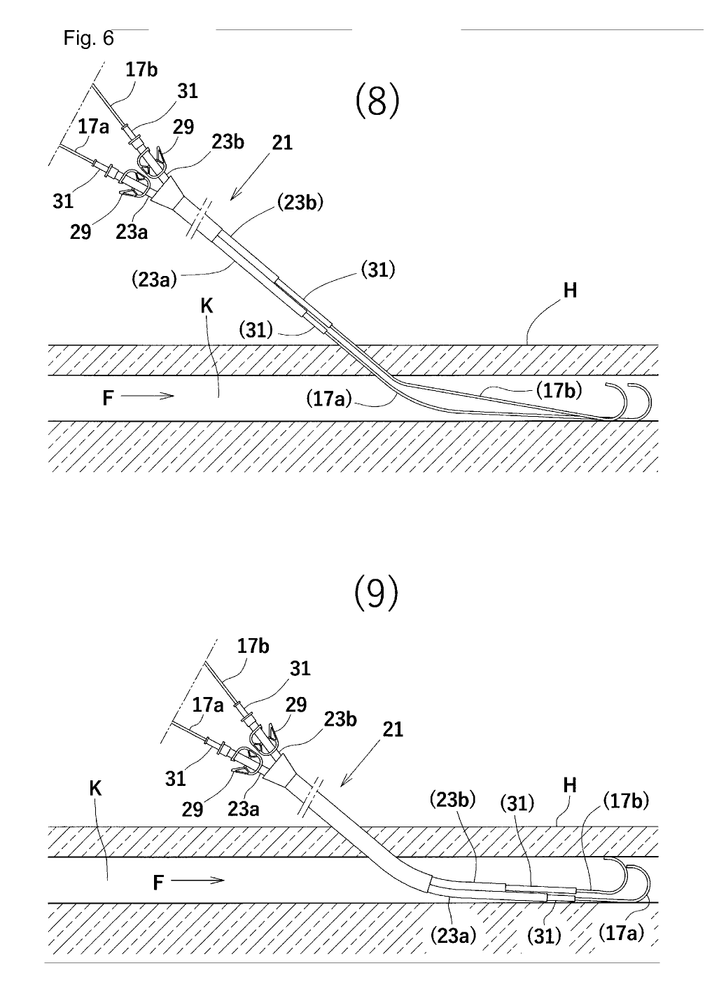

In FIG. 6, (8) is a view showing a state where the guidewires are respectively inserted into stylets that are inserted in the catheter in advance, and (9) is a view showing a state where the catheter is advanced along both the guidewires together with both the stylets until tip portions of both conduits of the catheter are inside the vessel.

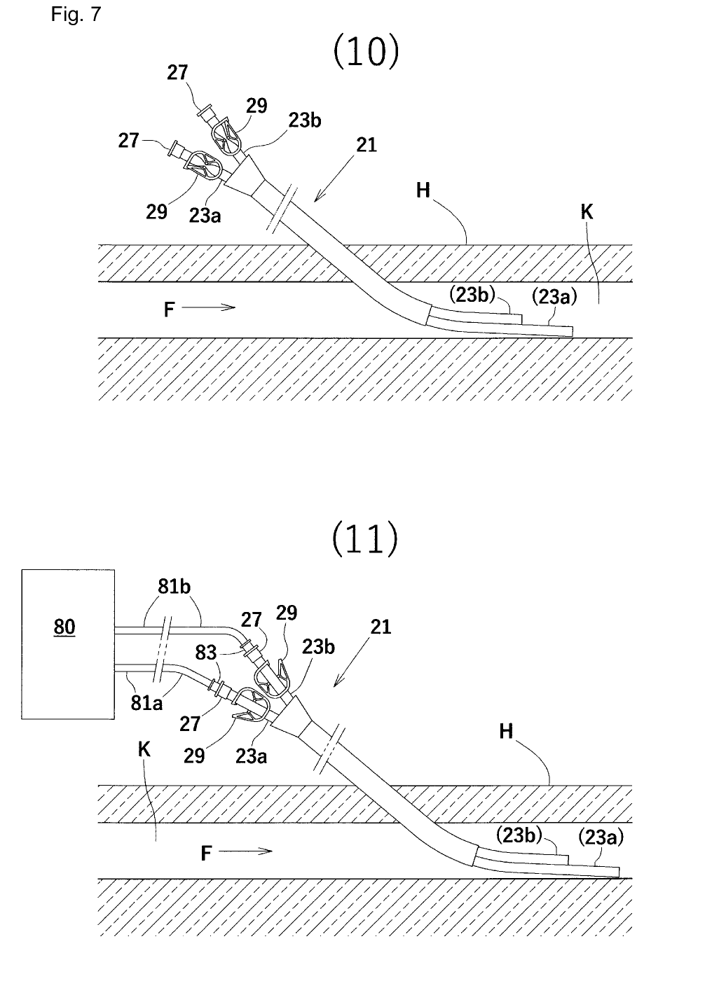

In FIG. 7, (10) is a view showing a state where both the guidewires and both the stylets are pulled out from the catheter, and both clips are put in an engaged state, and (11) is a view showing a state where the catheter is connected to a dialyzer, and both the clips are put in a disengaged state to perform treatment such as dialysis and purification of blood.

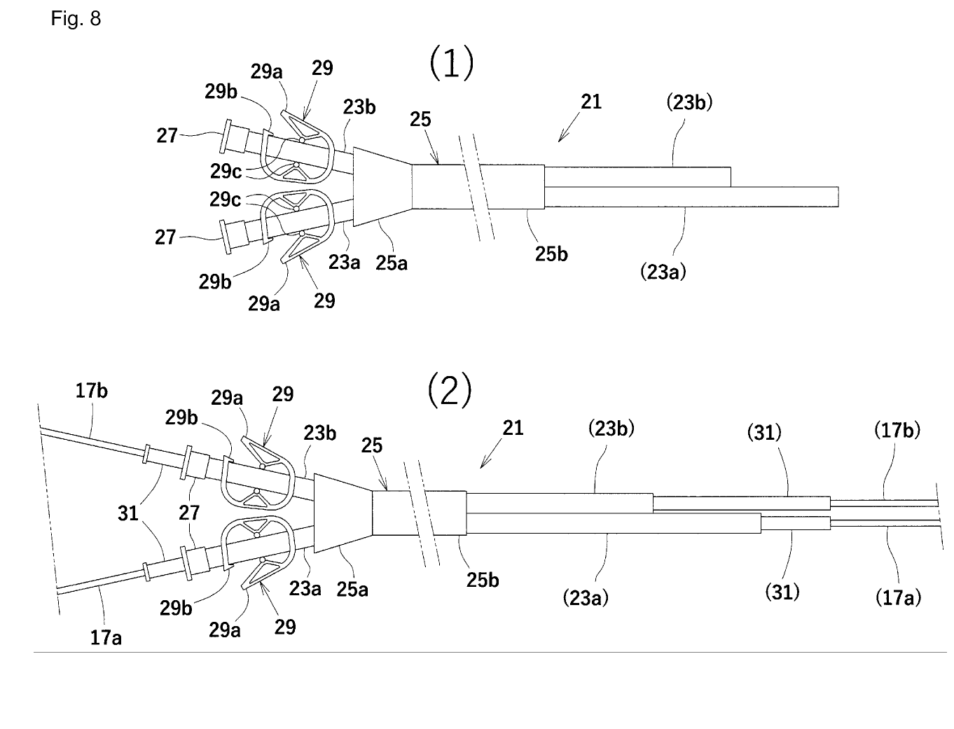

In FIG. 8, (1) is a side view of the catheter, and (2) is a view showing a state where the guidewire is inserted into the stylet that is inserted in the catheter.

In FIG. 9, (1) is a side view of a dilator according to a second embodiment of the present invention, and (2) is a sectional view along a line C-C in (1).

In FIG. 10, (1) is a side view of the dilator according to the third embodiment of the present invention, and (2) is a sectional view along a line D-D in (1).

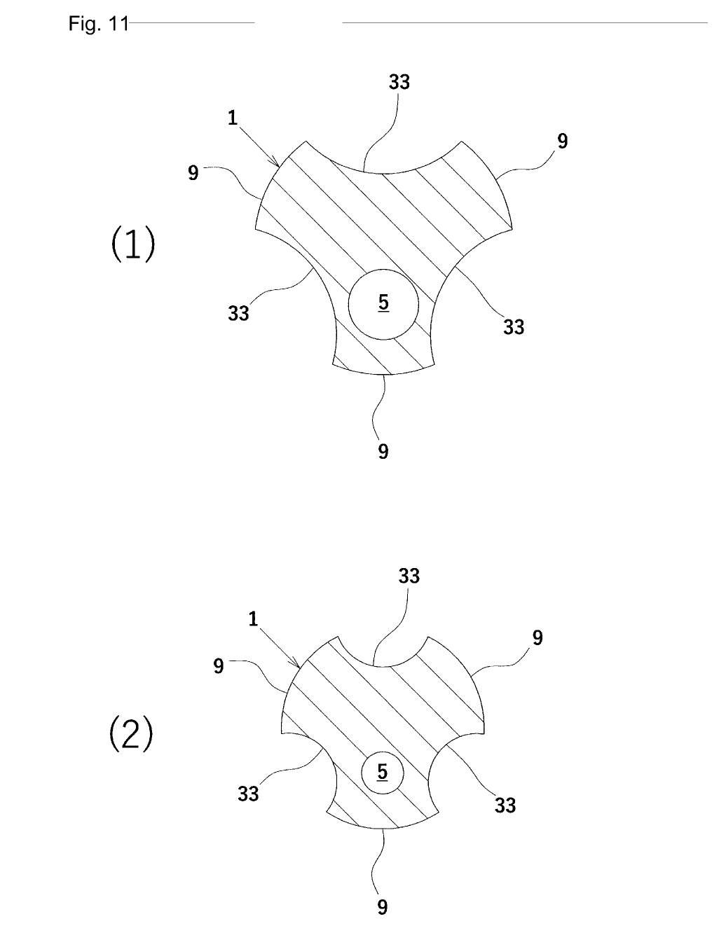

In FIG. 11, (1) is an enlarged end view along a line E-E in (1) of FIG. 10, and (2) is an enlarged end view along a line F-F in (1) of FIG. 10.

### DESCRIPTION OF THE PREFERRED EMBODIMENTS

### First Embodiment

A first embodiment according to the present invention will be described in detail with reference to accompanying drawings FIG. 1 through FIG. 8. Reference numeral 1 in (1) and (2) of FIG. 1 denotes an example of a dilator according to the present invention. The dilator 1 shown in these figures is used to dilate an opening formed in the skin by a puncture needle to facilitate the insertion of a catheter or used when inserting guidewires into a vessel, and is made from a stem-like member having a generally circular transverse section. The dilator 1 is made from resin having elasticity to some extent, for example, polypropylene resin, polyethylene resin, and nylon resin. The dilator 1 is provided with a first through-hole 5 pierced over an entire region of the dilator 1 from a distal end face 3a to a rear end face 3b in a longitudinal direction of the dilator 1, a second through-hole 7, which is a different through-hole from the first through-hole 5, pierced over a region of the dilator 1 from a midway to the rear end face 3b in the longitudinal direction of the dilator 1, and a tapered portion 9 formed such that it is tapered toward the distal end face 3a.

The first through-hole 5 and the second through-hole 7 are respectively sized to allow insertion of a first guidewire 17a and a second guidewire 17b, which will be mentioned later. The tapered portion 9 is formed generally in a conical shape, and is formed over the region starting at about halfway with respect to the entire length of the dilator 1 in the longitudinal direction thereof to the distal end face 3a. The portion of the dilator 1 in the longitudinal direction thereof other than the tapered portion 9 is formed such that a shape of an outside diameter of the transverse section over the entire region thereof is in a circular shape of generally the same size. Both ends of the first through-hole 5 are open on the distal end face 3a and the rear end face 3b of the dilator 1, respectively. The openings in the distal end face 3a and the rear end face 3b are denoted by reference numerals 5a and 5b, respectively in (1) and (2) of FIG. 2. The opening 5a on the distal end face 3a side is formed generally in a circular shape while the opening 5b on the rear end face 3b side is formed generally in a D-shape. Further, a cross-sectional area of the first through-hole 5 gradually increases from the distal end face 3a side toward the rear end face 3b side. Accordingly, the opening 5b is larger than the opening 5a.

One end of the second through-hole 7 located at the midway of the dilator 1 in the longitudinal direction thereof is open on a surface of the tapered portion 9 and forms an opening 7a, while the other end of the second through-hole 7 is open on the rear end face 3b of the dilator 1 and forms an opening 7b. A cross-sectional area of the second through-hole 7 is formed generally the same over the entire region of the second through-hole 7 in the longitudinal direction thereof. Note that, the cross-sectional area of the second through-hole 7 is not limited to this, and accordingly, the cross-sectional area of the second through-hole 7 may also be formed such that it gradually becomes small toward the opening 7a. The opening 7b of the second through-hole 7 is formed generally in a D-shape. The second through-hole 7 is formed such that it extends from the rear end face 3b in a direction along the axis of the dilator 1 and that it bends toward the opening 7a near the opening 7a in the tapered portion 9. In the present embodiment, the opening 7a is formed in a surface of the tapered portion 9 on the rear end face 3b side, however, the opening 7a may also be formed at an arbitrary location in the surface of the tapered portion 9 as long as the second through-hole 7 can be pierced.

When seen from a direction along the axis of the dilator 1, both the opening 7a which is one end of the second through-hole 7 and the opening 7b which is another end of the second through-hole 7 are biased to the same direction with respect to the axis of the dilator 1 (an upper direction in (2) of FIG. 1). Further, the size of the opening 5b of the first through-hole 5 and the size of the opening 7b of the second through-hole 7 in the rear end face 3b of the dilator 1 are different from each other. To be specific, as it can be understood from (2) of FIG. 2, the opening 5b of the first through-hole 5 is larger than the opening 7b of the second through-hole 7. Accordingly, the through-holes 5 and 7 can be distinguished from each other by merely looking at the respective openings 5b and 7b.

While the openings 5b and 7b were made to be of different sizes in the present embodiment, the two opening 5b and 7b may also be formed in different shapes instead. Further, the openings 5b and 7b may also be of different shapes and sizes. An example of forming the two opening 5b and 7b in different shapes includes a case in which the openings 5b and 7b are formed in combination of the two shapes selected from a generally D-shape, a generally circular shape, a generally rectangular shape, and a generally oval shape. By doing so, the through-holes 5 and 7 can be distinguished from one another by merely looking at the openings 5b and 7b. Further, a mark M is indicated on an outer peripheral surface of the dilator 1 near the rear end face 3b in proximity to the opening 7b. This, together with forming the openings 5b and 7b in different sizes, allows determination of which one of the openings 5b and 7b is the opening 7b of the second through-hole 7 easily by visual observation. Note that the position for indicating the mark M is not limited to the outer peripheral surface of the dilator 1, and the mark M may also be indicated on the rear end face 3b or on both the rear end face 3b and the outer peripheral surface as long as the opening 7b can be easily identified by merely looking at the mark M. Also, the relationship between a position of the opening 7a of the second through-hole 7 and the position indicated by the mark M is correlated in advance with the direction about the axis of the dilator 1. These positions are correlated such that the positions are located at the same positions with respect to the axis of the dilator 1. However, the positional relationship between the opening 7a and the mark M may also be such that the opening 7a and the mark M are located directly opposite to each other in the direction about the axis of the dilator 1.

### Procedure for Inserting the Catheter

A procedure for inserting a guidewire into a vessel using the above-mentioned dilator 1 and a procedure for inserting a catheter into a vessel of a human body using the guidewire inserted into the vessel in accordance with the procedure for inserting the guidewire will now be described in detail with reference to the accompanying FIGS. 3 through 8.

First, as shown in (1) of FIG. 3, a tip portion 11a of a puncture needle 11 of a syringe 15 constituted by a transparent syringe main body 13 and the puncture needle 11 attached to a tip portion of the syringe main body 13, the puncture needle 11 having a sharp tip and a hollow axial core portion, is stuck into a skin H of a human body, and the tip portion 11a is inserted into a vessel K. The puncture needle 11 is stuck into the skin H at an angle such that the tip portion 11a enters the vessel K in the same direction as a flow F of blood flowing in the vessel K when the tip portion 11a of the puncture needle 11 is inserted into the vessel K. While the vessel K may either be an artery or a vein, in general, it is easier to stick the puncture needle 11 into a vein than into an artery since the wall thickness of the vein is thinner than that of the artery. Therefore, the puncture needle 11 is desirably stuck into the vein. It can be determined that the tip portion 11a of the puncture needle 11 has entered the vessel K when entry of blood into the syringe main body 13 is observed.

Next, the syringe main body 13 is detached from the puncture needle 11 while keeping the tip portion 11a of the puncture needle 11 inserted in the vessel K. Then, a tip portion of the first guidewire 17a is inserted into a hollow space of the puncture needle 11 through an opening in the portion from which the syringe 13 had been detached (See (2) of FIG. 3). The first guidewire 17a includes a core wire, which is a thin stainless wire, and another thin stainless wire wound around the core wire, and both ends of the wire wound around the core wire are welded to both ends of the core wire to be integrated therewith. The wire wound around the core wire is wound with a certain minute clearance between adjacent wires, so that the first guidewire 17a is flexible enough to be freely flexed. The tip portion of the first guidewire 17a is curved into an arc shape so as not to damage the vessel K by the tip portion when the tip portion is inserted into the vessel K. The tip portion of the first guidewire 17a is inserted into the hollow space inside the puncture needle 11 in a linearly stretched out state. However, when the tip portion is further advanced through the hollow space inside the puncture needle 11 and inserted into the vessel K, the tip portion restores its original arc shape, eliminating the possibility of damaging the vessel K.

Next, as shown in (3) of FIG. 3, after an opening in the skin H formed by sticking with the tip portion 11a of the puncture needle 11 is dilated with a skin-cutting scalpel 19, as shown in (3) of FIG. 3, the dilator 1 is stuck over a rear end portion of the first guidewire 17a through the opening 5a in the distal end face 3a of the dilator 1 such that the first guidewire 17a is inserted in the first through-hole 5. At this time, a plug member 20 is attached to the opening 7b of the second through-hole 7 of the dilator 1 in advance to seal the opening 7b. Further, preferably, a hydrophilic lubricant coating agent is applied to the surface of the dilator 1 prior to insertion of the dilator 1. Hydrophilic lubricant coating agents include, for example, polyvinylpyrrolidone, maleic anhydride polymer, and cellulosic polymer. Such hydrophilic lubricant coating agents provide lubricity through wetting (water absorption), and reduce frictional resistance (sliding resistance) between the outer peripheral surface of the dilator 1 and the opening in the skin H or the opening in the vessel K into which the dilator 1 is inserted, allowing smooth insertion of the dilator 1 thereby.

Then, the dilator 1 is advanced along the longitudinal direction of the first guidewire 17a until both openings 5a and 7a of the dilator 1 are surely located inside the vessel K. At this time, the dilator 1 is advanced while visually checking such that the opening 7b and the mark M of the second through-hole 7 are located above the opening 5b of the first through-hole 5, so that the opening 7a will be directed toward the surface of the skin H when advancing of the dilator 1 has completed. Thus, the distal end face 3a side of the dilator 1 is inserted through the opening in the skin H until it is inside the vessel K (see (5) of FIG. 4). At this time, because the distal end face 3a side is tapered, the dilator 1 can be inserted easily into the opening in the skin H and into the opening in the vessel K through which the first guidewire 17a is inserted. Note that because the plug member 20 is attached to the opening 7b of the second through-hole 7 of the dilator 1, the blood will not flow out from the opening 7b.

Also, because the tapered portion 9 becomes thicker from the distal end face 3a side toward the rear end face 3b side, as the dilator 1 advances into the opening in the skin H and into the opening in the vessel K through which the first guidewire 17a is inserted, the opening in the skin H and the opening in the vessel K are dilated such that the dilator 1 can be easily inserted into the opening in the skin H and into the opening in the vessel K. Further, because the dilator 1 is made of a resinous material having flexibility to some extent, even if the first guidewire 17a is inserted inside the skin and the vessel K in a bent state, the dilator 1 will also be bent in accordance with the bent shape of the first guidewire 17a.

Then, the plug member 20 is removed from the opening 7b of the rear end face 3b of the dilator 1 inserted inside the vessel K, and the second guidewire 17b is inserted through the opening 7b to be inserted through the second through-hole 7. Further, the second guidewire 17b is inserted until it is inside the vessel K (see (6) of FIG. 5). Because the second through-hole 7 of the dilator 1 is substantially closed by the second guidewire 17b that has been inserted, only little blood flows from the opening 7 of the second through-hole 7.

The second guidewire 17b is made of a material similar to that of the first guidewire 17a and configured similarly to the first guidewire 17a, and the tip portion of the second guidewire 17b is inserted into the opening 7b of the dilator 1 in a linearly stretched out state. However, when the tip portion is further advanced through the through-hole 7 of the dilator 1 and inserted into the vessel K, the tip portion restores its original arc shape, eliminating the possibility of damaging the vessel K. Also, because the dilator 1 is advanced into the vessel K such that the opening 7a will be directed toward the surface side of the skin H, the opening 7a is not easily blocked by an inner wall of the vessel K. Accordingly, the second guidewire 17b is less likely to damage the vessel K when the second guidewire 17b is inserted into the vessel K.

After inserting the second guidewire 17b until it is inside the vessel K, the dilator 1 is pulled out from both the guidewires 17a and 17b while both the guidewires 17a and 17b are kept to be inserted in the vessel K (see (7) of FIG. 5).

Next, both the guidewires 17a and 17b are inserted into, for example, a catheter 21, as shown in (1) of FIG. 8. The catheter 21 is provided with a tubular first conduit 23a, a tubular second conduit 23b shorter than the first conduit 23a, a tubular hub 25 that integrally fastens these conduits 23a and 23b, female-side connectors 27 respectively attached to each one end of the conduits 23a and 23b, and clips 29 inserted into the conduits 23a and 23b at midway in the longitudinal direction thereof, that are located between the female-side connectors 27 and the hub 25. Materials for the first conduit 23a, the second conduit 23b, and the hub 25 include, for example, urethane resin, silicon resin, and vinyl chloride resin.

While the catheter 21 shown in FIG. 8 includes the first conduit 23a, the second conduit 23b, and the hub 25 as separate members, the configuration is not limited to this. The conduits 23a and 23b may be integrally molded with the hub 25, while portions of the conduits 23a and 23b protruding from the hub 25 may be configured separately, or the portions of the conduits 23a and 23b protruding from the hub 25 that are in contact with each other (the right-hand-side portion in (1) of FIG. 8 may be integrated.

A clip 29 is used for temporarily shutting off the fluid flow inside each of the conduits 23a and 23b. The clip 29 is configured such that, by engaging an engaging portion 29a of the clip 29 with an engaged portion 29b thereof, the respective conduits 23a and 23b are pinched between a pair of protrusions 29c and 29c of the clip 29, and the fluid flow inside the conduits 23a and 23b is shut off.

A diameter of the hub 25 on one end portion side, from which the respective rear end portions of the conduits 23a and 23b connected to the female-side connectors 27 protrude, is enlarged as the one end portion nears the female-side connectors 27. Lengths of the rear end portions of the conduits 23a and 23b that protrude from an enlarged-diameter portion 25a of the enlarged-diameter hub 25 are set to be generally the same, while the lengths of the tip portions of the conduits 23a and 23b that protrude from a small-diameter portion 25b of the hub 25 on an opposite side of the enlarged-diameter portion 25a are set such that the length of the second conduit 23b is shorter than that of the first conduit 23a.

When the catheter 21 is stuck over both the guidewires 17a and 17b, tubular stylets 31 (see (2) of FIG. 8) are preferably inserted into each of the conduits 23a and 23b of the catheter 21 in advance.

(8) of FIG. 6 shows a state where the guidewires 17a and 17b are respectively inserted into the stylets 31, from tip end sides of the respective stylets that have been inserted respectively into the conduits 23a and 23b of the catheter 21 in advance, and where the guidewires 17a and 17b protrude respectively from the rear ends of the respective stylets 31.

Next, the catheter 21 is advanced together with both the stylets 31 along the longitudinal direction of both the guidewires 17a and 17b, and the tip end portions of both the guidewires 23a and 23b are inserted through the opening in the skin H until the tip end portions 23a and 23b are inside the vein K (see (9) of FIG. 6). By inserting the stylets 31 into both the conduits 23a and 23b of the catheter 21 in advance, a rapid dimensional change between outside diameters of both the guidewires 17a and 17b and outside diameters of both the conduits 23a and 23b of the catheter 21 which would have otherwise occurred if the stylets 31 were not inserted can be replaced by a mild change. Further, in the case where both the conduits 23a and 23b of the catheter 21 have high flexibility, adequate rigidity will be exerted to the conduits 23a and 23b by inserting the stylets 31. As a result, the respective tip end portions of both the conduits 23a and 23b of the catheter 21 can be smoothly inserted through the opening in the skin H until inside the vessel K.

Note that, the above-mentioned hydrophilic lubricant coating agent is preferably applied to the surface of the catheter 21 and to the surfaces of the tip end portions of the stylets 31 protruding from the respective conduits 23a and 23b of the catheter 21 in advance. By doing so, frictional resistance (sliding resistance) induced between these outer peripheral surfaces and the opening of the skin H or the opening of the vessel K when inserting the catheter 21 together with the stylets 31 will be reduced, and therefore the catheter 21 can be inserted smoothly.

Next, both the guidewires 17a and 17b and both the stylets 31 are pulled out from both the conduits 23a and 23b of the catheter 21 while the tip end portions of both the conduits 23a and 23b of the catheter 21 are inserted inside the vessel K (see (10) of FIG. 7). Then, to prevent blood from flowing out from both the conduits 23a and 23b, the engaging portion 29a of the clip 29 is engaged with the engaged portion 29b of the clip 29 and the flow inside the conduits 23a and 23b is shut off.

Now, the insertion of catheter 21 into the vessel K is complete.

Next, an example of a method for conducting such treatment as dialysis and purification of blood in a state where both the conduits 23a and 23b of the catheter 21 are inserted inside the vessel K as described above, will be described with reference to (11) of FIG. 7. In (11) of FIG. 7, reference numeral 80 denotes a dialyzer, and one end of a drain pipe 81a that drains blood and one end of an intake pipe 81b that sucks blood are respectively connected to the dialyzer. On the other end portion of the drain pipe 81a and the intake pipe 81b, a male-side connector 83 is connected to each of the drain pipe 81a and the intake pipe 81b on an opposite side of the dialyzer. The dialyzer 80 and the catheter 21 are connected by coupling the female-side connector 27 of the catheter 21 to the respective male-side connecters 83. After the dialyzer 80 and the catheter 21 are connected, the engagement of the engaging portion 29a and the engaged portion 29b of the clip 29 is released, and blood is allowed to flow through the respective conduits 23a and 23b. Then, when a pump in the dialyzer 80 is actuated, blood inside the body is drawn into the dialyzer 80 via the second conduit 23b of the catheter 21 and the intake pipe 81b. The blood drawn in is then dialyzed and purified inside the dialyzer 80, and this blood is returned to the vessel K inside the body via the first conduit 23a of the catheter 21 and the drain pipe 81a. At this time, because an end portion of the second conduit 23b is located upstream of an end portion of the first conduit 23a as seen from a direction F, which is a direction of blood flow inside the vessel K, there is no possibility that the blood that has been dialyzed and purified inside the dialyzer 80 and has just been returned to inside the vessel K is sucked via the second conduit 23b.

After the treatment, that is, dialysis and purification of blood, has been conducted for a predetermined time and the treatment has been completed, the operation of the dialyzer 80 is stopped. At the same time, the engaging portion 29a of the clip 29 is engaged with the engaged portion 29b of the clip 29 to shut off the flow inside the conduits 23a and 23b, and the respective male-side connectors 83 are separated from the respective female-side connectors 27 of the catheter 21.

Next, to prevent blood inside the catheter 21 from clotting, a medicine filling apparatus (not shown) in which anticoagulant is stored is connected to the female-side connectors 27. Then, the engaging portion 29a of the clip 29 and the engaged portion 29b of the clip 29 are disengaged, and the anticoagulant is filled from the medicine filling apparatus into the catheter 21. The anticoagulant is a heparin, for example. After filling the anticoagulant into the catheter 21, the openings of the female-side connectors 27 are tightly plugged by plug members (not shown), and the conduits 23a and 23b of the catheter 21 are fixed by a fixing band (not shown) to the human body to prevent the conduits 23a and 23b from moving undesirably.

Now the treatment consisting of dialysis and purification of blood is complete.

As described above, with the dilator 1 according to the first embodiment of the present invention, because the first through-hole 5 and the second through-hole 7 are separately pierced in the dilator 1, the two guidewires 17a and 17b can be inserted into the through-holes 5 and 7, respectively. Accordingly, when the secondly inserted second guidewire 17b is inserted into the second through-hole 7 of the dilator 1, the secondly inserted second guidewire 17b can be smoothly inserted until it is inside the vessel K without being kinked with the firstly inserted first guidewire 17a that is inserted inside the first through-hole 5 in advance or by other incidents that hinder insertion of the second guidewire 17b.

Also, the dilator 1, which has been inserted until it is inside the vessel K for dilating the opening in the skin H and the opening in the vessel K to facilitate insertion of the catheter 21, can be used as is in this state to insert the secondly inserted second guidewire 17b until it is inside the vessel K. Thus, it is possible to insert the secondly inserted second guidewire 17b by using the dilator 1 without having to use the conventional introducer which is a set of a sheath and a dilator inserted therein. As a result, instrumental cost for insertion of the guidewire will be reduced, and at the same time, the insertion operation of the second guidewire 17b will be simplified due to the elimination of the operation for pulling out the dilator from the introducer. Further, because a conventional type introducer is not used, resistance due the step between the sheath and the dilator is not induced, and thus the dilator 1 can be smoothly inserted into the opening in the skin H and the opening in the vessel K.

Further, because the dilator 1 has a tapered portion 9 tapered toward the distal end face 3a, the dilator 1 can be easily inserted into the opening in the skin H and into the opening in the vessel K in which the firstly inserted first guidewire 17a is inserted, and at the same time the dilator 1 can dilate the opening in the skin H or the opening in the vessel K by the tapered portion 9 as the dilator 1 is advanced, which allows the dilator 1 to be smoothly inserted into the opening in the skin H and the opening in the vessel K.

In addition, because the opening 7a on one end of the second through-hole 7 located at the midway of the dilator 1 in the longitudinal direction thereof is open on the surface of the tapered portion 9, the diameter of the dilator 1 at which the opening 7a is located is thinner than parts other than the tapered portion 9. Accordingly, a gap tends to form between the part of the opening 7a of the second through-hole 7 and the inner wall of the vessel K, and the second guidewire 17b is less likely to damage the vessel K when the second guidewire 17b is inserted into the vessel K via the second through-hole 7. Also, even if a gap is not formed between the part of the opening 7a of the second through-hole 7 and the inner wall of the vessel K, adhesion force between the part of the opening 7a of the second through-hole 7 and the inner wall of the vessel K can be reduced because the dilator 1 is thinned at the part of the opening 7a. As a result, it is less likely to damage the vessel K by the second guidewire 1 when the second guidewire 17b is inserted into the vessel K via the second through-hole 7.

Also, because the opening 7a on one end and the opening 7b on the other end of the second through-hole 7 are provided biased to the same direction with respect to the axis of the dilator 1, when the dilator 1 is inserted until it is inside the vessel K, the insertion is conducted by visually checking such that the position of the opening 7b of the second through-hole 7 in the rear end face 3b of the dilator 1 is at a desired position, and accordingly, the opening 7a of the second through-hole 7 located inside the vessel K can be positioned at a desired position in a circumferential direction inside the vessel K.

Further, the sizes of the respective openings 5b and 7b of the first through-hole 5 and the second through-hole 7 in the rear end face 3b of the dilator 1 are different from each other, the first through-hole 5 can be easily distinguished from the second through-hole 7 by merely looking at the respective openings 5b and 7b.

Still further, because the relationship between the position of the opening 7a of the second through-hole 7 located at the midway of the dilator 1 in the longitudinal direction thereof and the position indicated by the mark M is correlated in advance with the direction about the axis of the dilator 1, by visually checking such that the position of the mark M is at a desired position when inserting the dilator 1 until it is inside the vessel K, the opening 7a of the second through-hole 7 located inside the vessel K can be positioned inside the vessel K at desired position in the circumferential direction thereof.

Now, two other embodiments will be described hereunder as embodiments other than the one described above. Note that, a member, a portion, and the like that are the same or similar to those described in the first embodiment will be denoted by the same reference numerals, and a detailed description thereof is omitted. It would be understood that the same or similar configurations as those explained in the above-mentioned embodiment or in other embodiments described later operate in a similar manner and thus have a similar effect.

### Second Embodiment

In the above-mentioned first embodiment, an example was shown in which the opening 7a of the second through-hole 7 is provided in the surface of the tapered portion 9 of the dilator 1. However, as in the second embodiment shown in FIG. 9, only one recessed portion 33 may be formed in a part of the surface of the tapered portion 9 by recessing one location thereof and the opening 7a may be formed in the recessed portion 33. The recessed portion 33 is formed to extend long in a direction along the axis of the dilator 1.

According to the dilator 1 of the second embodiment, because the recessed portion 33 is formed by recessing a part of the surface of the dilator 1 excluding the distal end face 3a and the rear end face 3b, and the one end of the second through-hole 7 is open in this recessed portion 33, it is less likely to damage the inner wall of the vessel K by corner portions of the opening 7a.

Note that, in this second embodiment, the opening 7a of the second through-hole 7 is formed at a part near the rear end face 3b of the recessed portion 33, however, the opening 7a may be formed at an arbitrary location in the recessed portion 33 as long as the second through-hole 7 can be pierced.

### Third Embodiment

In the example shown for the above-mentioned second embodiment, only one recessed portion 33 was formed and the opening 7a of the second through-hole 7 was formed in this recessed portion 33. However, three recessed portions 33, as in the third embodiment shown in FIG. 10 and FIG. 11 may be provided, and the opening 7a of the second through-hole 7 may be formed in one of the three recessed portions 33. Each recessed portion 33 is formed as a long groove extending in a direction along the axis of the dilator 1, and the recessed portions 33 are formed in the surface of the tapered portion 9 at equivalent angles and at a certain distance apart from each other in the circumferential direction. In the third embodiment, while each of the recessed portions 33 was formed in some region in a direction along the axis of the dilator 1, the invention is not limited thereto, and the recessed portions 33 may also be provided along the entire region from the distal end face 3a to the rear end face 3b of the dilator 1.

According to the dilator 1 of the third embodiment, because the recessed portion 33 is formed as a long groove extending in a direction along the axis of the dilator 1 and because three recessed portions 33 are formed in the surface of the dilator 1 at equivalent angles and at a certain distance apart from each other in the circumferential direction thereof, a contact area between the tapered portion 9 of the dilator 1 and the opening in the skin H and the opening in the vessel K becomes small at the time when the tapered portion 9 of the dilator 1 is inserted into the opening in the skin H or the opening in the vessel K. Consequently, the resistance induced during insertion of the dilator 1 can be reduced.

Also, because the opening 7a of the second through-hole 7 is open within one of the three recessed portions 33, the chances of damaging the inner wall of the vessel K by the corner portions of the opening 7a are reduced.

Note that, in this third embodiment, the opening 7a of the second through-hole 7 is formed at a part near the rear end face 3b of the recessed portion 33, however, the opening 7a may be formed at an arbitrary location in the recessed portion 33 as long as the second through-hole 7 can be pierced.

In addition, in the example shown for the third embodiment, three recessed portions 33 were formed in the surface of the tapered portion 9 of the dilator 1. However, two, four, or more recessed portions 33 may be formed, and the opening 7a of the second through-hole 7 may be formed in one of a plurality of recessed portions 33.

The above-mentioned first to third embodiments of the invention are examples for explaining the present invention, and therefore the present invention is not limited to each of the above-mentioned embodiments. Accordingly, various changes can be made to the present invention, as appropriate, without departing from the spirit and scope of the claims and specification. It should be noted that such modified dilator 1 is also included within a technical scope of the present invention.

For example, in each of the above-mentioned embodiments, an example was shown in which the opening 7a of the second through-hole 7 is formed in the surface of the tapered portion 9. Instead, the opening 7a may be formed in a surface of the dilator 1 other than the surface of the tapered portion 9 excluding the distal end face 3a and the rear end face 3b (for example, a surface near the tapered portion 9). Also, the opening 7a may be formed across the surface of the tapered portion 9 and the surface of the dilator 1 other than the tapered portion 9. In the case of such a modified example, a gap is less likely to be formed between the part of the opening 7a and the inner wall of the vessel K compared to a case where the opening 7a is formed in the tapered portion 9. The gap should be more easily generated if the dilator is 1 is inserted while visually checking that the opening 7b of the second through-hole 7 comes to a desired position. Also, the adhesion force between the opening 7a portion and the inner wall of the vessel K may be reduced. In the case of such a modified example, at least a part of the recessed portions 33 according to the above-mentioned second embodiment and third embodiment will be formed in a surface other than the tapered portion 9.

Further, in each of the above-mentioned embodiments, an example was shown in which the mark M was indicated on the dilator 1. However, the mark M is not limited to the above and may also be formed by coextruding resin to form the dilator 1 and forming the mark M with the resin material during the coextrusion. To be specific, resin material of a different color from the resin material for the dilator 1 is continuously mixed by small amounts into the resin material for the dilator 1 to be coextruded, and during solidification of the resin material, the resin material of a different color solidifies at a location biased with respect to the axis of the dilator 1. Thus, the mark M is formed inherently by the resin material of a different color from the resin material for the dilator 1. The mark M formed in this way is formed partially in cross-section and along an entire region of the dilator 1 in the longitudinal direction thereof.

In the case of forming the dilator 1 by coextruding resin, the dilator 1 is formed as a long stem-like material having the first through-hole 5 and the second through-hole 7. Then, the formed long stem-like material is cut into appropriate length pieces, and an outer peripheral surface on one end portion thereof is heated and pressurized so as to reduce the diameter and form the tapered portion 9. At this time, cross-sectional areas of both the through-holes 5 and 7 located inside the tapered portion 9 will be reduced. Even after the diameter is reduced, the first guidewire 17a can be inserted into the first through-hole 5, however, the diameter is reduced to such a size that the second guidewire 17b cannot be inserted into the second through-hole 7 which is smaller than the first through-hole 5. Accordingly, it is necessary to form the opening 7a of the second through-hole 7 to make the second through-hole 7 communicate with the outside by piercing a hole with a piercing tool in the outer peripheral surface of the dilator 1 excluding the distal end face 3a and the rear end face 3b. At this time, a position for piercing the hole (a position on the outer peripheral surface of the dilator 1 in a circumferential direction thereof) needs to be determined, and if the resin material that constitutes the mark M is positioned near the second through-hole 7 and if the resin material is coextruded such that a part thereof is exposed on the surface of the dilator 1, the mark M may be used as a marker that indicates the position for piercing the hole. In this case, the mark M does not need to be formed on the entire region of the dilator 1 in the longitudinal direction thereof, but instead, the mark M may be provided at least in a region from the rear end face 3b of the dilator 1 to a position scheduled to be formed with the opening 7a of the second through-hole 7.

What is claimed is:

1. A stem-like dilator for assisting insertion of a catheter into a vessel, comprising:
a first through-hole pierced over an entire region of the dilator from a distal end face to a rear end face in the longitudinal direction of the dilator;
a second through-hole, which is a different through-hole from the first through-hole, pierced over a region of the dilator from a midway to the rear end face in the longitudinal direction of the dilator; and
a tapered portion formed such that it is tapered toward the distal end face, wherein
the first through-hole and the second through-hole are sized to allow insertion of flexible guidewires for assisting insertion of the catheter.

2. The dilator according to claim 1, wherein one end of the second through-hole located at the midway of the dilator in the longitudinal direction thereof is open on a surface of the tapered portion while the other end of the second through-hole is open on the rear end face.

3. The dilator according to claim 1, wherein when seen from a direction along the axis of the dilator, an opening in the one end of the second through-hole and an opening in the other end of the second through-hole are biased to the same direction with respect to the axis of the dilator.

4. The dilator according to claim 1, wherein the opening in the rear end face of the first through-hole is different from the opening in the rear end face of the second through-hole in at least one of a shape and a size thereof.

5. The dilator according to claim 1, wherein a mark that is observable from outside is indicated on a region including at least one of the rear end face and the proximity to the rear end face, and
a positional relationship between a position of the opening on one end of the second through-hole located at the midway of the dilator in the longitudinal direction thereof and a position indicated by the mark is correlated in advance with the direction about the axis of the dilator.

6. The dilator according to claim 1, wherein a recessed portion is formed by recessing a part of the surface of the dilator excluding the distal end face and the rear end face, and the one end of the second through-hole is open in the recessed portion.

7. The dilator according to claim 6, wherein
the recessed portion is formed as a long groove extending in a direction along the axis of the dilator, and a plurality of recessed portions are formed in the surface of the dilator at a certain interval in a circumferential direction thereof, and
the one end of the second through-hole is open in one of the plurality of recessed portions.
